SEQUENCE LISTING (11 sequences)

SEQ ID NO: 1 - protein, 10 aa, synthetic construct
GFSLTSYGVH

SEQ ID NO: 2 - protein, 16 aa, synthetic construct
VIYADGSTNYNPSLKS

SEQ ID NO: 3 - protein, 12 aa, synthetic construct
ARAYGNYWYIDV

SEQ ID NO: 4 - protein, 11 aa, synthetic construct
KSSESVSNDVA

SEQ ID NO: 5 - protein, 7 aa, synthetic construct
YAFHRFT

SEQ ID NO: 6 - protein, 9 aa, synthetic construct
HQAYSSPYT

SEQ ID NO: 7 - protein, 118 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 8 - protein, 107 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 9 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 10 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 11 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0084168 A1
(43) Pub. Date: Mar. 13, 2025

(54) STABLE HIGH CONCENTRATION SODIUM CHLORIDE FORMULATIONS CONTAINING PD-1 ANTIBODY AND METHODS OF USE THEREOF

(71) Applicant: BeiGene Switzerland GmbH, Basel (CH)

(72) Inventors: Jun WU, Suzhou, (CN); Xiaoqing JIN, Suzhou, (CN); Yu JI, Suzhou, (CN); Sufang GU, Suzhou, (CN); Jian SHEN, Suzhou, (CN); Bo QIU, Guangzhou, (CN)

(73) Assignee: BeiGene Switzerland GmbH, Basel (CH)

(21) Appl. No.: 18/910,935

(22) Filed: Oct. 9, 2024

Related U.S. Application Data
(63) Continuation of application No. PCT/CN2023/087860, filed on Apr. 12, 2023.

(30) Foreign Application Priority Data
Apr. 14, 2022 (WO) PCT/CN2022/086868

(51) Int. Cl. C07K 16/28; A61K 39/00

(52) U.S. Cl. CPC C07K 16/2818; A61K 2039/505; A61K 2039/55511; C07K 2317/24; C07K 2317/94

(57) ABSTRACT

The present invention relates generally to the field of pharmaceutical formulations of antibodies against human programmed death receptor PD-1, or antigen binding fragments thereof. The formulations may further contain a histidine buffer, an inorganic salt, a sugar polyol, and a non-ionic surfactant. The pharmaceutical formulations of the present invention exhibit low viscosity and a substantial degree of antibody stability after being subjected to thermal and other physical stress. Also provided are methods of making and methods of using such antibody formulations.

### CROSS-REFERENCE TO RELATED APPLICATIONS

This application is a continuation of International Patent Application No. PCT/CN2023/087860, filed Apr. 12, 2023, which claims priority from International Patent Application No. PCT/CN2022/086868, filed Apr. 14, 2022.

### SEQUENCE LISTING

The instant application contains a Sequence Listing which has been submitted electronically in xml format, and is hereby incorporated by reference in its entirety. Said xml file was created on Sep. 18, 2024, is named 138881_0876_Sequence_Listing.xml, and is 17 bytes in size.

### FIELD OF THE DISCLOSURE

Disclosed herein are stable high concentration formulations comprising antibodies or antigen binding fragments thereof that binds to human programmed death receptor 1 (PD-1). Also disclosed herein are methods of preparing the formulations and treating cancers with the formulations of the present disclosure.

### BACKGROUND OF THE DISCLOSURE

Antibodies as therapeutics have seen increased use in the clinic. However, while antibodies generally have a similar structure, they are different in primary amino acid sequence, even for antibodies that bind to the same target protein. The characteristics of the primary amino acid sequence of the antibody is one of the major determinants of the properties of antibody solubility and/or stability in different formulations. An antibody formulation that provides for solubility and stability of one antibody can perform poorly for another antibody, resulting in antibody precipitation or fragmentation. This is especially true when a subcutaneous antibody formulation is desired.

Subcutaneous injection has gained increasing attention for the delivery of protein therapeutics due to its potential to provide for patient self-administration. With a fast, low-volume injection, the patient can administer the antibody therapeutic without the need for an intravenous infusion, which typically requires a hospital visit. However, many antibodies require a certain dose to be effective, generally requiring concentration of the antibody into a small volume. The volume limitation of subcutaneous route of administration is a critical factor to be considered for subcutaneous administration, leading to a need for highly concentrated antibody dose. In turn, this creates challenges relating to solubility, physical and chemical stability of the protein, difficulties of manufacture, storage, and delivery of the subcutaneous antibody formulation. For example, antibodies can lose solubility and form particulates in certain formulations during processing and/or storage, which renders the subcutaneous administration less effective. Due to the concentrated nature of the antibody in a subcutaneous formulation, high viscosity is another problem to overcome as it limits the injectability of the product. Also, in the manufacturing process, a highly viscous antibody formulation presents difficulties in processing, particularly in ultrafiltration and sterile filtration. Lastly, the subcutaneous antibody formulation needs to maintain the structure and function of the antibody. A subcutaneous antibody formulation that leads to proteolysis or degradation of the antibody structure will have reduced efficacy as well as one that impairs the antibody's ability to bind to its target protein.

Thus, there is a long felt need in the art for subcutaneous antibody formulations of anti-human PD-1 antibodies for treating various cancers and infectious diseases. Such formulations can have good antibody solubility, stability, a long shelf-life, and be amenable to administration at high concentrations.

### SUMMARY OF THE INVENTION

The disclosure provides a stable, low viscosity and high concentration antibody formulation. A low viscosity pharmaceutical formulation comprising:
about 10 mg/mL to about 200 mg/mL of an anti-programmed death receptor 1 (PD-1) antibody, or antigen binding fragment thereof;
a formulation buffer providing a pH of about 5.0 to about 7.0;
a sugar polyol;
a viscosity reducer; and
a non-ionic surfactant,
wherein the pharmaceutical formulation has a viscosity of no more than 30 cP, and an osmolarity from about 200 mOsmol/kg to about 400 mOsmol/kg.

The formulation wherein the PD-1 antibody or antigen binding fragment thereof, comprises a heavy chain variable region (a) a HCDR1 (Heavy Chain Complementarity Determining Region 1) of SEQ ID NO: 1, (b) a HCDR2 of SEQ ID NO:2, (c) a HCDR3 of SEQ ID NO:3 and a light chain variable region that comprises: (d) a LCDR1 (Light Chain Complementarity Determining Region 1) of SEQ ID NO:4, (e) a LCDR2 of SEQ ID NO:5, and (f) a LCDR3 of SEQ ID NO:6.

The formulation wherein the formulation buffer is selected from the group consisting of histidine, acetate, citrate, succinate, phosphate, mixture of histidine and acetic acid, or mixture of histidine and citric acid

The formulation wherein the formulation buffer is histidine.

The formulation wherein the concentration of buffer is 15 mM to 25 mM.

The formulation wherein the formulation comprises 20 mM histidine buffer.

The formulation wherein the pH is 5.5-6.0.

The formulation wherein the sugar polyol is selected from the group consisting of trehalose, sucrose, sorbitol, mannitol, maltose, dextran, or (2-hydroxypropyl)-β-cyclodextrin.

The formulation wherein the sugar polyol is trehalose.

The formulation wherein the trehalose concentration is from 70 mM to 240 mM.

The formulation wherein the trehalose concentration is from 80 mM to 160 mM.

The formulation wherein the trehalose concentration is from 70 mM to 100 mM.

The formulation wherein the trehalose concentration is 80 mM.

The formulation wherein the viscosity reducer is an inorganic salt selected from the group consisting of sodium chloride, magnesium chloride, calcium chloride, sodium acetate, sodium sulfate, ammonium chloride or ammonium sulfate.

The formulation wherein the inorganic salt is sodium chloride at a concentration of 50 mM to 150 mM,

The formulation wherein the sodium chloride is at a concentration of 50 mM to 100 mM.

The formulation wherein the sodium chloride concentration is 70 mM

The formulation wherein the non-ionic surfactant is selected from the group consisting of polysorbate 20, polysorbate 80 or poloxamer 188.

The formulation wherein the concentration of polysorbate 20 is from 0.02% to 0.08%.

The formulation wherein polysorbate 20 concentration is 0.08%.

The formulation wherein the formulation comprises 20 mM Histidine-Histidine HCl, 100 mM NaCl, 70 mM trehalose and 0.08% polysorbate 20, with a pH of pH6.0.

The formulation wherein the formulation comprises 20 mM Histidine-Histidine HCl, 50 mM NaCl, 100 mM trehalose and 0.02% polysorbate 20, with a pH of pH6.0.

The formulation wherein the formulation comprises 20 mM Histidine-Histidine HCl, 70 mM NaCl, 80 mM trehalose and 0.08% polysorbate 20 with a pH of pH6.0.

The formulation wherein the concentration of the anti-human PD-1 antibody, or antigen binding fragment thereof is from about 10 mg/mL to 200 mg/mL.

A method of making an antibody formulation, the method comprising:
a. adding trehalose and sodium chloride to the antibody to achieve an antibody formulation having a concentration of trehalose no less than 50 mM and a concentration of sodium chloride no less than 25 mM, wherein the antibody comprises a heavy chain variable region (a) a HCDR1 (Heavy Chain Complementarity Determining Region 1) of SEQ ID NO: 1, (b) a HCDR2 of SEQ ID NO:2, (c) a HCDR3 of SEQ ID NO:3 and a light chain variable region that comprises: (d) a LCDR1 (Light Chain Complementarity Determining Region 1) of SEQ ID NO:4, (e) a LCDR2 of SEQ ID NO:5, and (f) a LCDR3 of SEQ ID NO:6;
b. concentrating the antibody formulation of (a) from 150-200 mg/mL; and
c. adding polysorbate 20 to the antibody formulation of (b) to achieve an antibody formulation having a concentration of polysorbate 20 of no less than 0.01 mg/mL, wherein the antibody formulation of (c) is in an aqueous solution and has a viscosity of no more than 30 cP at 25° C., and wherein the antibody formulation of (c) is stable upon agitation, freeze-thaw and thermal stress.

A method for treating cancer in a human patient in need thereof comprising subcutaneous administration of an effective amount of an anti-human PD-1 antibody formulation.

The method wherein the anti-human PD-1 antibody formulation is subcutaneously administered at a dose of about 100 mg to about 1000 mg.

The method wherein the anti-human PD-1 antibody formulation is subcutaneously administered at a dose of 200 mg.

The method wherein the anti-human PD-1 antibody formulation is subcutaneously administered at a dose of 300 mg.

The method wherein the anti-human PD-1 antibody formulation is subcutaneously administered at a dose of 400 mg.

The method wherein the anti-human PD-1 antibody formulation is subcutaneously administered at a dose of 500 mg.

The method wherein the anti-human PD-1 antibody formulation is subcutaneously administered once a week.

The method wherein the anti-human PD-1 antibody formulation is subcutaneously administered once every 2 weeks.

The method wherein the anti-human PD-1 antibody formulation is subcutaneously administered once every 3 weeks.

The method wherein the cancer is lung cancer (including small-cell lung cancer, or non-small cell lung cancer), adrenal cancer, liver cancer, stomach cancer, cervical cancer, melanoma, renal cancer, breast cancer, colorectal cancer, leukemia, bladder cancer, bone cancer, brain cancer, an endometrial cancer, head and neck cancer, lymphoma, ovarian cancer, skin cancer, thyroid tumor, or esophageal cancer.

The method wherein the human patient is administered at least one other therapeutic is zanubrutinib, pamiparib, an anti-CTLA4 antibody, an anti-4-1BB antibody, an anti-OX40 antibody, an anti-TIGIT antibody, an anti-TIM-3 antibody, a CD40 agonist, a TLR agonist, a CAR-T cell, or a chemotherapeutic agent.

In some embodiments, the antibody formulation comprises an anti-PD-1 antibody, or antigen binding fragment thereof, a formulation buffer, a sugar polyol, a viscosity reducer, and a non-ionic surfactant. In some embodiments, the formulation buffer provides a pH range of between 5.0 and 7.0. In some embodiments, the antibody formulation has a viscosity of no more than 30 centiPoise (cP). In some embodiments, the antibody formulation has an osmolarity of about 200 mOsmol/kg to about 400 mOsmol/kg. In some embodiments, the antibody formulation is stable upon agitation, freeze-thaw and thermal stress.

In some embodiments, the antibody formulation can comprise between about 10 mg/mL to about 200 mg/mL anti-PD-1 antibody or antigen binding fragment thereof, a formulation buffer, a sugar polyol, a viscosity reducer, and a non-ionic surfactant, and has a pH of about 6.0±0.5. In some embodiments, the antibody formulation can consist essentially of about 100 mg/mL to about 180 mg/mL anti-PD-1 antibody, a formulation buffer, a sugar polyol, a viscosity reducer, and a non-ionic surfactant, and has a pH of 6.0±0.5.

In some embodiments, the formulation buffer is selected from the group consisting of histidine, acetate, citrate, succinate, phosphate, mixture of histidine and acetic acid, mixture of histidine and citric acid. In some embodiments, the formulation buffer can be histidine buffer. In some embodiments, the concentration of histidine buffer is from about 10 mM to about 30 mM. In some embodiments, the concentration of the histidine buffer is about 20 mM histidine.

In some embodiments, the sugar polyol is selected from the group consisting of trehalose, sucrose, sorbitol, mannitol, maltose, dextran, or (2-hydroxypropyl)-β-cyclodextrin. In some embodiments, the sugar polyol can be trehalose. In some embodiments the trehalose is a, α-trehalose dihydrate. In other embodiments, the sugar polyol can be sucrose. In some embodiments, the concentration of sugar polyol can be from about 25 mM to about 240 mM. In some embodiments, the concentration of sugar polyol can be from about 50 mM to about 150 mM, preferably about 70 mM, about 75 mM, about 80 mM, about 85 mM, about 90 mM, about 95 mM, or about 100 mM.

In some embodiments, the viscosity reducer is inorganic salt. In some embodiments, the viscosity reducer is selected from the group consisting of sodium chloride, magnesium chloride, calcium chloride, sodium acetate, sodium sulfate, ammonium chloride or ammonium sulfate. In some embodiments, the viscosity reducer is sodium chloride. In some embodiments, the concentration of sodium chloride can be from about 25 mM to about 150 mM, about 50 mM, about 55 mM, about 60 mM, about 65 mM, about 70 mM, about 75 mM, about 80 mM, about 85 mM, about 90 mM, about 95 mM, or about 100 mM.

In some embodiments, the non-ionic surfactant is selected from the group consisting of polysorbate 80 (PS80), polysorbate 20 (PS20) or poloxamer188. In some embodiments, the concentration of non-ionic surfactant can be from about 0.01 to about 1 mg/mL. In some embodiments, the concentration of polysorbate is about 0.2 mg/mL, about 0.3 mg/mL, about 0.4 mg/mL, about 0.5 mg/mL, about 0.6 mg/mL, about 0.7 mg/mL, or about 0.8 mg/mL. In some embodiments, the polysorbate is polysorbate 20.

In some embodiments, the antibody formulation comprises about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL or about 200 mg/mL of an anti-PD-1 antibody, or antigen binding fragment thereof, about 20 mM histidine buffer, about 70 mM to about 100 mM α, α-trehalose dihydrate or sucrose, about 50 mM to about 100 mM sodium chloride, about 0.2 mg/mL to about 0.8 mg/mL polysorbate 20, and the antibody formulation is of a pH 6.0±0.5. In some embodiments, the antibody formulation has a viscosity of no more than 30 cP at 25° C.

In some embodiments of the invention the anti-PD-1 antibody is Tislelizumab (BGB-A317) or an antigen binding fragment of Tislelizumab.

Also provided herein are methods of making a stable, low viscosity antibody formulation, the method comprising: adding trehalose or sucrose and sodium chloride to the antibody to achieve an antibody formulation having a concentration of trehalose or sucrose no less than 50 mM and a concentration of sodium chloride no less than 25 mM, concentrating the antibody to about 200 mg/mL; adding polysorbate 20 to the antibody formulation to achieve an antibody formulation comprising a concentration of polysorbate 20 of no less than 0.01 mg/mL. wherein the antibody formulation is an aqueous solution and has a viscosity of no more than 30 cP at 25° C.

Also provided herein are methods of treating cancer in a human patient who has cancer, comprising subcutaneous administration to the patient an effective amount of the PD-1 antibody formulation as described herein.

Provided herein are methods of treating cancer in a human patient who has a PDL-1 expressing cancer, comprising subcutaneous administration to the patient an effective amount of the PD-1 antibody formulation as described herein.

### BRIEF DESCRIPTION OF THE DRAWINGS

FIG. 1A-B shows the results of SEC-HPLC study (FIG. 1A) and CZE study (FIG. 1B) for 10 mg/ml Tislelizumab formulations stored at 40° C. for 2 weeks (noted as “40C2W” in graphs) and exposed to light for 2 weeks (noted as “pho2W” in graphs). TO refers to the initial point of samples.

FIG. 2A-B shows the amounts of aggregates (FIG. 2A) and monomer (FIG. 2B) of each of the formulations after freeze/thaw (noted as “3FT” in graphs), shaking (noted as “SK” in graphs) and thermal stress (noted as “40C4W” in graphs) as measured by SEC-HPLC. TO refers to the initial point of samples. “25C6M” indicates that the formulation was stored at 25° C. for 6 months. “5C6M” indicates that the formulation was stored at 5° C. for 6 months.

FIG. 3 shows the results of an CZE study of the subcutaneous antibody formulations over a 4-week period storage at 40° C. (noted as “40C4W” in graphs). TO refers to the initial timepoint. “25C6M” indicates that the formulation was stored at 25° C. for 6 months. “5C6M” indicates that the formulation was stored at 5° C. for 6 months.

FIG. 4 shows the purity of the formulations at 40° C. over a 4-week period (noted as “40C4W” in graphs) as measured by CE-SDS under non-reduced conditions. TO refers to the initial timepoint. “25C6M” indicates that the formulation was stored at 25° C. for 6 months. “5C6M” indicates that the formulation was stored at 5° C. for 6 months.

FIG. 5A-D shows the results of SEC-HPLC study (FIG. 5A and FIG. 5B), CZE study (FIG. 5C) and CE-SDS(NR) study (FIG. 5D) for formulation F18 stored at 25° C. (noted as “25C”) and 5±3° C. (noted as “5C”) conditions. “0” month refers to the initial time point of samples.

FIG. 6A-B shows the data of bioavailability of the F18 subcutaneous formulation in a Sprague Dawley rat model.

FIG. 7 shows the bioavailability data of the F18 subcutaneous formulation in a monkey model.

FIG. 8 are photographs showing there is no injection site reaction in both rats and minipigs when administering the subcutaneous formulation at various sites on the animal.

### DETAILED DESCRIPTION OF THE DISCLOSURE

### Definitions

Unless specifically defined elsewhere in this document, all other technical and scientific terms used herein have the meaning commonly understood by one of ordinary skill in the art.

As used herein, including the appended claims, the singular forms of words such as “a,” “an,” and “the” include their corresponding plural references unless the context clearly dictates otherwise.

The term “or” is used to mean, and is used interchangeably with, the term “and/or” unless the context clearly dictates otherwise.

Throughout this specification and the claims which follow, unless the context requires otherwise, the word “comprise,” and variations such as “comprises” and “comprising,” will be understood to imply the inclusion of a stated amino acid sequence, DNA sequence, step or group thereof, but not the exclusion of any other amino acid sequence, DNA sequence, step. When used herein the term “comprising” can be substituted with the term “containing,” “including” or sometimes “having.”

The terms “administration,” “administering,” “treating,” and “treatment” herein, when applied to an animal, human, experimental subject, cell, tissue, organ, or biological fluid, means contact of an exogenous pharmaceutical, therapeutic, diagnostic agent, or antibody formulation to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both.

The term “therapeutically effective amount” as herein used, refers to the amount of an anti-PD-1 antibody that, when administered to a subject for treating a disease, or at least one of the clinical symptoms of a disease or disorder, is sufficient to effect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition. In some embodiment of present disclosure, the subject is a human.

“Pharmaceutical formulation” or “formulation” refers to antibody preparations which are in such form as to allow the active ingredients to be effective, and which contain no additional components that would be toxic to the subjects to which the formulation would be administered.

A “stable” formulation is one in which the antibody is prepared in such a way as to preserve the antibody's physical stability and/or chemical stability and/or biological activity over time. Various analytical techniques for measuring protein stability are available in the art and are reviewed in Peptide and Protein Drug Delivery, 247-301, Vincent Lee Ed., Marcel Dekker, Inc. New York, N.Y., Pubs. (1991) and Jones, A. Adv. Drug Delivery Rev. 10:29-90?(1993). Stability can be measured at a selected temperature for a selected time period.

The term “antibody” herein is used in the broadest sense and specifically covers antibodies (including full length monoclonal antibodies) and antibody fragments so long as they recognize antigen, e.g., PD-1. An antibody is usually monospecific, but may also be described as idiospecific, heterospecific, or polyspecific. Antibody molecules bind by means of specific binding sites to specific antigenic determinants or epitopes on antigens.

The term “monoclonal antibody” or “mAb” or “Mab” herein means a population of substantially homogeneous antibodies, i.e., the antibody molecules comprised in the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) may be obtained by methods known to those skilled in the art. See, for example Kohler G et al., Nature 1975 256:495-497; U.S. Pat. No. 4,376,110; Ausubel F M et al., CURRENT PROTOCOLS IN MOLECULAR BIOLOGY 1992; Harlow E et al., ANTIBODIES: A LABORATORY MANUAL, Cold spring Harbor Laboratory 1988; and Colligan J E et al., CURRENT PROTOCOLS IN IMMUNOLOGY 1993. The mAbs disclosed herein may be of any immunoglobulin class including IgG, IgM, IgD, IgE, IgA, and any subclass thereof. A hybridoma producing a mAb may be cultivated in vitro or in vivo. High titers of mAbs can be obtained by in vivo production where cells from the individual hybridomas are injected intraperitoneally into mice, such as pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called “complementarity determining regions (CDRs)”, which are located between relatively conserved framework regions (FR). The CDRs are usually aligned by the framework regions, enabling binding to a specific epitope. In general, from N-terminal to C-terminal, both light and heavy chain variable domains sequentially comprise FR-1 (or FR1), CDR-1 (or CDR1), FR-2 (FR2), CDR-2 (CDR2), FR-3 (or FR3), CDR-3 (CDR3), and FR-4 (or FR4). The assignment of amino acids to each domain is, generally, in accordance with the definitions of Sequences of Proteins of Immunological Interest, Kabat, et al., National Institutes of Health, Bethesda, Md.; 5th ed.; NIH Publ. No. 91-3242 (1991); Kabat (1978) Adv. Prot. Chem. 32: 1-75; Kabat, et al., (1977) J. Biol. Chem. 252:6609-6616; Chothia, et al, (1987) J Mol. Biol. 196:901-917 or Chothia, et al., (1989) Nature 342:878-883.

The term “hypervariable region” means the amino acid residues of an antibody that are responsible for antigen-binding. The hypervariable region comprises amino acid residues from a “CDR” (i.e., VL-CDR1, VL-CDR2 and VL-CDR3 in the light chain variable domain and VH-CDR1, VH-CDR2 and VH-CDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues mean those variable domain residues other than the hypervariable region residues defined herein as CDR residues.

Unless otherwise indicated, “antibody fragment” or “antigen-binding fragment” means antigen binding fragments of antibodies, i.e., antibody fragments that retain the ability to bind specifically to the antigen bound by the full-length antibody, e.g., fragments that retain one or more CDR regions. Examples of antigen binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

An antibody that binds to a specified target protein with specificity is also described as specifically binding to a specified target protein. This means the antibody exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies or binding fragments thereof, useful in the present invention will bind to the target protein with an affinity that is at least two-fold greater, preferably at least 10-times greater, more preferably at least 20-times greater, and most preferably at least 100-times greater than the affinity with non-target proteins. An antibody herein is said to bind specifically to a polypeptide comprising a given amino acid sequence.

The term “human antibody” herein means an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” means an antibody that comprises only mouse or rat immunoglobulin protein sequences, respectively.

The term “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The antibody of the present application has potential therapeutic uses in treating cancer. The term “cancer” or “tumor” herein means or describes the physiological condition in mammals that is typically characterized by unregulated cell growth. Examples of cancer include but are not limited to, lung cancer (including small-cell lung cancer, or non-small cell lung cancer), adrenal cancer, liver cancer, stomach cancer, cervical cancer, melanoma, renal cancer, breast cancer, colorectal cancer, leukemia, bladder cancer, bone cancer, brain cancer, an endometrial cancer, head and neck cancer, lymphoma, ovarian cancer, skin cancer, thyroid tumor, or esophageal cancer.

Further, the antibody of the present application has potential therapeutic uses in controlling viral infections and other human diseases that are mechanistically involved in immune tolerance or “exhaustion.” In the context of the present application, the term “exhaustion” refers to a process which leads to a depleted ability of immune cells to respond to a cancer or a chronic viral infection.

### Anti-PD-1 Antibody

The present disclosure provides for anti-PD-1 antibodies and subcutaneous formulations thereof. For example, Tislelizumab (BGB-A317), is an anti-PD-1 antibody disclosed in U.S. Pat. No. 8,735,553 with the sequences provided in Table 1 below.

TABLE 1
Tislelizumab (BGB-A317)

| | SEQ ID | |
|---|---|---|
| Domain | NO: | Amino Acid Sequence |
| HCDR1 | SEQ ID | GFSLTSYGVH |
| | NO: 1 | |
| HCDR2 | SEQ ID | VIYADGSTNYNPSLKS |
| | NO: 2 | |
| HCDR3 | SEQ ID | ARAYGNYWYIDV |
| | NO: 3 | |
| LCDR1 | SEQ ID | KSSESVSNDVA |
| | NO: 4 | |
| LCDR2 | SEQ ID | YAFHRFT |
| | NO: 5 | |
| LCDR3 | SEQ ID | HQAYSSPYT |
| | NO: 6 | |
| VH | SEQ ID | QVQLQESGPGLVKPSETLSLTCTVS |
| | NO: 7 | GFSLTSYGVHWIRQPPGKGLEWIGV |
| | | IYADGSTNYNPSLKSRVTISKDTSK |
| | | NQVSLKLSSVTAADTAVYYCARAYG |
| | | NYWYIDVWGQGTTVTVSS |
| VL | SEQ ID | DIVMTQSPDSLAVSLGERATINCKS |
| | NO: 8 | SESVSNDVAWYQQKPGQPPKLLINY |
| | | AFHRFTGVPDRFSGSGYGTDFTLTI |
| | | SSLQAEDVAVYYCHQAYSSPYTFGQ |
| | | GTKLEIK |
| IgG4 | SEQ ID | ASTKGPSVFPLAPCSRSTSESTAAL |
| constant | NO: 9 | GCLVKDYFPEPVTVSWNSGALTSGV |
| domain | | HTFPAVLQSSGLYSLSSVVTVPSSS |
| | | LGTKTYTCNVDHKPSNTKVDKRVES |
| | | KYGPPCPPCPAPPVAGGPSVFLFPP |
| | | KPKDTLMISRTPEVTCVVVAVSQED |
| | | PEVQFNWYVDGVEVHNAKTKPREEQ |
| | | FNSTYRVVSVLTVVHQDWLNGKEYK |
| | | CKVSNKGLPSSIEKTISKAKGQPRE |
| | | PQVYTLPPSQEEMTKNQVSLTCLVK |
| | | GFYPSDIAVEWESNGQPENNYKTTP |
| | | PVLDSDGSFFLYSKLTVDKSRWQEG |
| | | NVFSCSVMHEALHNHYTQKSLSLSL |
| | | GK |
| Heavy | SEQ ID | QVQLQESGPGLVKPSETLSLTCTVS |
| Chain | NO: 10 | GFSLTSYGVHWIRQPPGKGLEWIGV |
| | | IYADGSTNYNPSLKSRVTISKDTSK |
| | | NQVSLKLSSVTAADTAVYYCARAYG |
| | | NYWYIDVWGQGTTVTVSSASTKGPS |
| | | VFPLAPCSRSTSESTAALGCLVKDY |
| | | FPEPVTVSWNSGALTSGVHTFPAVL |
| | | QSSGLYSLSSVVTVPSSSLGTKTYT |
| | | CNVDHKPSNTKVDKRVESKYGPPCP |
| | | PCPAPPVAGGPSVFLFPPKPKDTLM |
| | | ISRTPEVTCVVVAVSQEDPEVQFNW |
| | | YVDGVEVHNAKTKPREEQFNSTYRV |
| | | VSVLTVVHQDWLNGKEYKCKVSNKG |
| | | LPSSIEKTISKAKGQPREPQVYTLP |
| | | PSQEEMTKNQVSLTCLVKGFYPSDI |
| | | AVEWESNGQPENNYKTTPPVLDSDG |
| | | SFFLYSKLTVDKSRWQEGNVFSCSV |
| | | MHEALHNHYTQKSLSLSLGK |
| Light | SEQ ID | DIVMTQSPDSLAVSLGERATINCKS |
| chain | NO: 11 | SESVSNDVAWYQQKPGQPPKLLINY |
| | | AFHRFTGVPDRFSGSGYGTDFTLTI |
| | | SSLQAEDVAVYYCHQAYSSPYTFGQ |
| | | GTKLEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |

Anti-PD1 antibodies can include, without limitation, Tislelizumab, Pembrolizumab or Nivolumab. Tislelizumab is disclosed in U.S. Pat. No. 8,735,553. Pembrolizumab (formerly MK-3475), as disclosed by Merck, in U.S. Pat. Nos. 8,354,509 and 8,900,587 is a humanized IgG4-K immunoglobulin which targets the PD 1 receptor and inhibits binding of the PD 1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human lgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma.

### Further Alteration of the Framework of Fc Region

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are altered to thereby alter the ability of the antibody to fix complement. This approach is described in, e.g., the PCT Publication WO94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the PCT Publication WO00/42072 by Presta. Moreover, the binding sites on human IgG1 for FcγRI, FcγRII, FcγRIII and FcRn have been mapped and variants with improved binding have been described (see Shields et al., J. Biol. Chem. 276:6591-6604, 2001).

In still another aspect, the glycosylation of an antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such a glycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). PCT Publication WO 99/54342 by Umana et al., describes cell lines engineered to express glycoprotein-modifying glycosyl transferases (e.g., beta (1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G L, et al., 2010 MAbs, 2:181-189). On the other hand, natural IgG4 was found less stable in stress conditions such as in acidic buffer or under increasing temperature (Angal, S. 1993 Mol Immunol, 30:105-108; Dall′Acqua, W. et al, 1998 Biochemistry, 37:9266-9273; Aalberse et al., 2002 Immunol, 105:9-19). Reduced ADCC can be achieved by operably linking the antibody to IgG4 engineered with combinations of alterations to have reduced or null FcγR binding or C1q binding activities, thereby reducing or eliminating ADCC and CDC effector functions. Considering physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and 7Fc region were reported to have impact on antibody interaction with Fcγ receptors (Chappel S M, et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al, 2000 Nature Medicine, 6:443-446; Arnold J. N., 2007 Annu Rev immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate PD-1 antibodies with low ADCC, CDC and instability, it is possible to modify the hinge and Fc region of human IgG4 and introduce a number of alterations. These modified IgG4 Fc molecules can be found disclosed in SEQ ID NOs: 83-88, U.S. Pat. No. 8,735,553.

### Methods of Treatment

The antibodies or antigen-binding fragments of the present disclosure are useful in a variety of applications including, but not limited to, methods for the treatment of an PD-1-associated disorder or disease. In one aspect, the PD-1-associated disorder or disease is cancer.

In one aspect, the present disclosure provides a method of treating cancer. In certain aspects, the method comprises administering to a patient in need an effective amount of an anti-PD-1 antibody or antigen-binding fragment. The cancer can include, without limitation, lung cancer (including small-cell lung cancer, or non-small cell lung cancer), adrenal cancer, liver cancer, stomach cancer, cervical cancer, melanoma, renal cancer, breast cancer, colorectal cancer, leukemia, bladder cancer, bone cancer, brain cancer, an endometrial cancer, head and neck cancer, lymphoma, ovarian cancer, skin cancer, thyroid tumor, or esophageal cancer.

An antibody or antigen-binding fragment of the invention can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or antigen-binding fragment of the invention will depend on the type of disease to be treated, the type of antibody, the severity and course of the disease, whether the antibody is administered for preventive or therapeutic purposes, previous therapy, the patient's clinical history and response to the antibody, and the discretion of the attending physician.

Antibodies that are directed to PD-1 have been shown to be safe when administered to human cancer patients in various dose ranges and administration cycles. The subcutaneous antibody formulations disclosed herein can be administered at 100 mg, 200 mg, 300 mg, 400 mg, 500 mg, 600 mg, 700 mg, 800 mg, 900 mg or 1000 mg. The subcutaneous antibody formulation can be administered twice per day, daily, once per week, twice per week, three times per week, four times per week, five times per week, once every two weeks, once every three weeks, once every month, once every two months, once every three months, once every four months, once every five months or once every six months. In some embodiments, the dosing regimen comprises administering Tislelizumab at 100 mg once every three weeks. In some embodiments, the dosing regimen comprises administering Tislelizumab at 200 mg once every three weeks. In some embodiments, the dosing regimen comprises administering Tislelizumab at 300 mg once every three weeks. In some embodiments, the dosing regimen comprises administering Tislelizumab at 400 mg once every three weeks. In some embodiments, the dosing regimen comprises administering Tislelizumab at 500 mg once every three weeks. In some embodiments, the dosing regimen comprises administering Tislelizumab at 600 mg once every three weeks.

In certain embodiments, Tislelizumab can be administered in combination with other therapies, for example, zanubrutinib, pamiparib, an anti-CTLA4 antibody, an anti-4-1BB antibody, an anti-OX40 antibody, an anti-TIGIT antibody, an anti-TIM-3 antibody, a CD40 agonist, a TLR agonist, a CAR-T cell, or a chemotherapeutic agent.

### Pharmaceutical Compositions and Formulations

Also provided are compositions, including pharmaceutical formulations, comprising an anti-PD-1 antibody or antigen-binding fragment thereof. In certain embodiments, compositions comprise one or more antibodies or antigen-binding fragments that bind to PD-1. These compositions can further comprise suitable carriers, such as pharmaceutically acceptable excipients including buffers.

Pharmaceutical formulations of an anti-PD-1 antibody or antigen-binding fragment as described herein are prepared by mixing such antibody or antigen-binding fragment having the desired degree of purity with one or more pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG) or polysorbate 20.

The disclosure provides for various pharmaceutical formulations of an anti-PD1 antibody as described in detail herein. As an example, the formulation can comprise an anti-PD1 antibody, a buffer which provides a certain pH (e.g., histidine), a sugar polyol (e.g., sucrose or trehalose), a viscosity reducer (e.g., NaCl) and a non-ionic surfactant (e.g., polysorbate 20).

Sustained-release preparations can be prepared. Suitable examples of sustained-release preparations include semipermeable matrices of solid hydrophobic polymers containing the antibody, which matrices are in the form of shaped articles, e.g., films, or microcapsules.

The formulations to be used for in vivo administration are generally sterile. Sterility can be readily accomplished, e.g., by filtration through sterile filtration membranes.

### Examples

The examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entirety.

### Analytical Methods

This methods section provides a summary of the methods used in the following Examples 1-5.

### SEC-HPLC

Formation of soluble aggregates is analyzed by size exclusion chromatography (SEC) on a Waters HPLC system. Protein is separated based on molecular size on a TSKgel G3000™ SWXL column maintained at 37±5° C. using an isocratic gradient. Molecular weight species are eluted and detected by UV absorption at 280 nm. The distribution of aggregates, monomer and fragments are quantitated via the peak areas for standards and samples.

### CZE

The charge heterogeneity of a sample is determined using PA800 Plus™ (Beckman) by a capillary zone electrophoresis method (CZE) also known as free solution capillary electrophoresis. Samples are separated based on their electrophoretic mobilities caused by differences in charge and hydrodynamic radius of the analytes in a capillary filled with a buffer solution containing caproic acid. The samples are analyzed in their native state when an external electric field is applied resulting in a specific peak pattern showing the various charge variants of the antibody (acidic, basic and main charge variants). Samples are injected by pressure and the mobilized proteins are detected by UV absorbance at 214 nm.

### CE-SDS(NR)

The purity of sample is determined using PA800 Plus™ (Beckman) by a capillary gel electrophoresis (CE) method. Samples are denatured with sodium dodecyl sulphate (SDS) and separated based on size in a capillary filled with a gel that acts as a sieving medium. In non-reduced (NR) samples, an alkylating agent, N-Ethylmaleimide (NEM), is added to avoid any fragmentation induced by sample preparation and to ensure that the main IgG peak remains intact. Samples are injected electrokinetically and the mobilized proteins are detected by UV absorbance at 200 nm using a UV detector. The reportable value for non-reduced samples is the time corrected area percent (TCA) % of the IgG main peak.

### Protein Concentration

Protein concentrations are determined at UV 280 nm.

### Viscosity

The viscosity of the antibody formulations is measured on a chip-based microVISC™ instrument (Rheosense), in which the pressure difference correlates with solution dynamic viscosity. Sample size is approximately 70-100 μL. Aliquots are loaded into a 400 μL microVISC™ disposable pipette and connected to the chip. Triplicate measurements are taken at a shear rate of 500 S⁻¹ and at a temperature of about 25° C.

### Thermal Stability

Determining stability was performed using Uncle™ (Unchained Labs) which combines 3 different measurement modes—fluorescence, Static Light Scattering (SLS) and Dynamic Light Scattering (DLS). SLS and intrinsic fluorescence were conducted to determine the temperature of on-set aggregation (Tagg) and melting temperature (Tm) of formulations, respectively.

### Turbidity

UV absorption at 350 nm is measured using 96 well plate Molecular Devices M2E™ reader as an indication of turbidity. The absorption readings are controlled against blank well reading and normalized for sample pathlength.

### Visible Particles

Visible particles were examined against a black background and a white background using white fluorescent light about 2000 lux. The vial under inspection was gently swirled and inspected for no less than 5 seconds against each of the backgrounds.

### Subvisible Particles

Subvisible particles were analyzed using micro-flow imaging (MFI, Micro-Flow Imaging™ 5200, ProteinSimple). Water flushes were performed between each analysis. Besides, water flush was performed before sample analysis to ensure the background counts were appropriate for testing.

The average cumulative counts per mL were reported.

### Example 1: Impact of pH on the Stability of Low Concentration PD-1 Antibody Formulations

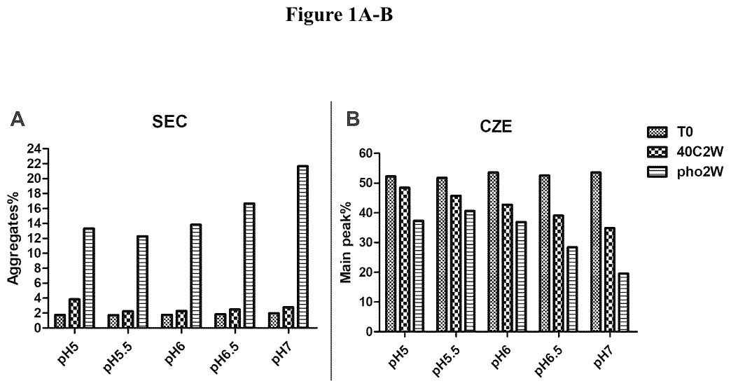

In order to determine the stability of the antibody within a pH range, Tislelizumab (Table 1) was prepared and purified. At first, low concentration of antibody (10 mg/ml) were prepared by dialyzing Tislelizumab into 20 mM disodium hydrogen phosphate-citric acid buffer at different pH (5.0, 5.5, 6.0, 6.5 and 7.0) with 10 kDa MWCO dialysis cassettes. This antibody solution was filtered by Millex™ GP 0.22 μm PES 33 mm filter and filled into 2 mL ready-to-use glass vials (Schott). To evaluate the effect on antibody stability at low concentrations, antibody samples were placed in a 40° C. stability chamber for two weeks (noted as “40C2W” in FIG. 1A-B) and then exposed to light in a 10° C. stability chamber for two weeks (noted as “pho2W” in FIG. 1A-B). As a control, the sample was assayed at its initial time point, noted as TO in the graphs. Purity by SEC-HPLC and charge heterogeneity by CZE were measured for all samples. Results are shown graphically in FIGS. 1A-B.

A clear trend in pH-dependent charge profile was observed in which lower pH formulations demonstrated higher main peak % both under thermal stress and light stress. The highest antibody aggregates were observed in formulations at pH 5.0 under thermal stress and under light stress at pH 7.0. Taken together, the SEC-HPLC results (FIG. 1A) and the CZE results (FIG. 1B), demonstrated that low concentration anti-PD-1 antibody formulations at pH 5.5-6.5 were relatively more stable than at the pH values of 5.0 and 7.0. Thus, it could be concluded that a pH range of pH 5.5-6.5 provided for stability of the antibody.

### Example 2: Impact of pH on the Viscosity and Stability of High Concentration PD-1 Antibody Formulations

With the pH range determined at a low concentration of antibody, this data was used in experiments for high concentrations of antibody. To prepare Tislelizumab stock solutions, Tislelizumab was buffer exchanged into the different buffers listed in Table 2 with 10 kDa MWCO dialysis cassettes. To prepare the trehalose-containing (150 mM) high concentration formulations, high concentration trehalose stock solutions were spiked into Tislelizumab stock solutions, and then concentrated to about 150 mg/ml of Tislelizumab by using 30 kDa Amicon Ultra™ centrifugal filters.

Protein concentration and viscosity were measured. Results are shown in Table 2. The results showed that formulations formulated in Histidine-Histidine HCl buffer at pH 5.5 and pH 6.0 demonstrated the lowest viscosity. Concentrations of 150 mg/ml of Tislelizumab in the same Histidine-Histidine HCl buffer, but at pH higher than 6.0 resulted in higher viscosity. As shown in Table 2, Tislelizumab formulations F4 and F5 provided good results around good viscosity and low aggregation. Tislelizumab antibody formulation F5 was of note with a viscosity (cP) of 15.53 and aggregation of 2.99.

To determine the freeze-thaw stability of high concentration (about 150 mg/mL) formulations of Tislelizumab, each of the formulated Tislelizumab solutions were filtered by Millex™ GP 0.22 μm PES 33 mm filter and filled into 2 mL glass vials. The samples were subjected to three cycles of freezing at −40° C. and thawing at room temperature (Freeze-thaw (3FT). In addition, samples were stored at 40° C. for 2 weeks (40C2W). Aggregation formation was evaluated by SEC-HPLC. As a control, the samples were analyzed at its initial time point, noted as TO in the table. The results are summarized in Table 2. The SEC-HPLC results showed that similar amounts of aggregates were formed across the spectrum of formulations tested in the freeze-thaw experiments, with most of the values between the initial timepoint and the end of the freeze-thaw being identical. This indicates that these Tislelizumab antibody formulations provided appropriate stability to the antibody at high concentrations. With regard to the Tislelizumab antibody formulations stored at 40° C. for 2 weeks (40C2W), about a 0.6Fe to 150 increase in aggregates were detected, indicating again that these formulations provided appropriate stability to Tislelizumab antibody at high concentration. The lowest amounts of aggregates were observed with Formulation 4 (F4) over the 2-weeks storage at 40° C. and Formulation 5 (F5) for the freeze-thaw cycles.

Considering both aggregation and solution viscosity, high concentration anti-PD-1 antibody formulations comprising Histidine-Histidine HCl (particularly pH 6.0) buffer produced the best results.

TABLE 2
Viscosity and Aggregates Formation of High Concentration
Formulations of PD-1 antibody in Different Buffers

| | | | | % Aggregates by | | |
|---|---|---|---|---|---|---|
| | | Antibody | | SEC-HPLC | | |
| | | concentration | Viscosity | | | Freeze- |
| No. | Formulation | (mg/mL) | (cP) | T0 | 40C2W | Thaw(3FT) |
| F1 | pH6.5, 15 mM Histidine + 25 mM | 155.31 | 35.61 | 1.99 | 3.36 | 1.99 |
| | Citrate, 150 mM Trehalose | | | | | |
| F2 | pH6.0, 15 mM Histidine + 25 mM | 159.81 | 36.48 | 1.97 | 3.15 | 1.96 |
| | Citrate, 150 mM Trehalose | | | | | |
| F3 | pH6.5, 20 mM Histidine-Histidine | 154.26 | 45.50 | 2.08 | 2.97 | 1.91 |
| | HCl, 150 mM Trehalose | | | | | |
| F4 | pH6.0, 20 mM Histidine-Histidine | 149.38 | 28.81 | 1.93 | 2.61 | 1.92 |
| | HCl, 150 mM Trehalose | | | | | |
| F5 | pH5.5, 20 mM Histidine-Histidine | 144.09 | 15.53 | 1.88 | 2.99 | 1.88 |
| | HCl, 150 mM Trehalose | | | | | |
| F6 | pH6.0, 20 mM Histidine-HAc, | 158.58 | 39.81 | 1.96 | 3.00 | 1.93 |
| | 150 mM Trehalose | | | | | |
| F7 | pH5.5, 20 mM Histidine-HAc, | 155.19 | 26.66 | 1.94 | 3.07 | 1.92 |
| | 150 mM Trehalose | | | | | |
| F8 | pH6.0, 20 mM Histidine-Citric acid, | 155.70 | 32.16 | 1.94 | 2.98 | 1.92 |
| | 150 mM Trehalose | | | | | |
| F9 | pH5.5, 20 mM NaAc-HAc, | 155.19 | 29.15 | 2.15 | 3.49 | 2.13 |
| | 150 mM Trehalose | | | | | |

Notes:
T0 refers to the initial time point of samples. 40C2W refers to the samples upon two-weeks storage at 40° C. Freeze-Thaw (3FT) refers to the samples upon three cycles freezing and thawing.

### Example 3: Evaluation of the Conformational and Colloidal Stability of Low Concentration PD-1 Antibody Formulations Comprising NaCl

This experiment determined the effect of pH and NaCl on the conformational and colloidal stability of anti-PD-1 antibody. In this study, varying concentrations of NaCl and trehalose stock solutions in 20 mM Histidine-Histidine HCl buffer at pH 5.5, 6.0 and 6.5 were prepared. Subsequently, Tislelizumab was buffer exchanged into 20 mM Histidine-Histidine HCl buffer (pH 5.5, 6.0 and 6.5) by dialysis to prepare Tislelizumab stock solutions. Trehalose stock solutions and/or NaCl stock solutions were spiked into Tislelizumab stock solutions to achieve the desired target excipient concentration (Table 3). The final antibody concentration of each sample was adjusted to 10 mg/ml.

The conformation and colloidal stability of Tislelizumab in the formulations containing NaCl (Table 3) were evaluated by measuring the midpoint unfolding temperature (Tm) and the temperature of on-set aggregation (Tagg). Tm and Tagg values showed a clear downward trend with increasing NaCl concentration, while a slight upward trend with increasing pH. Addition of 150 mM NaCl significantly decreased the conformation and colloidal stability of Tislelizumab formulations. These results indicate that at low antibody concentrations, Tislelizumab formulations at pH 5.5-6.0 with 50-100 mM NaCl demonstrated best conformation and colloidal stability.

TABLE 3
Midpoint Unfolding and Aggregation Temperatures of
Low Concentration Anti-PD-1 Antibody Formulations

| | Formulations | | | | | |
|---|---|---|---|---|---|---|
| | | | Trehalose | Thermal Properties | | |
| | | NaCl | Dihydrate | | | Tagg |
| No. | pH | (mM) | (mM) | Tm1(° C.) | Tm2(° C.) | (473 nm, ° C.) |
| F10 | 5.5 | 50 | 160 | 57.32 | 80.10 | 77.87 |
| F11 | 5.5 | 150 | None | 54.71 | 77 | 74.70 |
| F12 | 6.0 | 100 | 80 | 59.76 | 79.54 | 77.16 |
| F13 | 6.5 | 50 | 160 | 64.12 | 81.47 | 79.14 |
| F14 | 6.5 | 150 | None | 61.28 | 79.09 | 76.73 |

### Example 4: Viscosity of High Concentration PD-1 Antibody Formulations

This experiment determined the effects of varying concentrations of trehalose and NaCl on the viscosity of high concentration anti-PD-1 antibody formulations. In this study, varying concentrations of NaCl and trehalose stock solutions in 20 mM Histidine-Histidine HCl buffer at pH 6.0 were prepared. Subsequently, Tislelizumab was buffer exchanged into 20 mM Histidine-Histidine HCl buffer (pH 6.0) by dialysis to prepare high concentration Tislelizumab stock solutions. Trehalose stock solutions and/or NaCl stock solutions were spiked into Tislelizumab high concentration antibody stock solutions (Table 4 and Table 5). The samples were concentrated to varying concentrations by using 30 kDa Amicon Ultra™ centrifugal filters.

Viscosity analysis was performed at a flow rate of 500 S⁻¹ and at the temperature of about 25° C. Results are shown in Table 4. This data indicates that the viscosity of anti-PD-1 antibody formulations increased exponentially with the increase of antibody concentration. Compared with base buffer formulations, addition of 100 mM trehalose and 240 mM trehalose increased the viscosity, both at a concentration of 100 mM trehalose and especially at the higher concentration of 240 mM trehalose. Subcutaneous formulations have an element of “syringe-ability” which is the ability of the subcutaneous formulation to be administered through a syringe (e.g., 20-25 gauge) needle. Thus, addition of viscosity reducer was necessary.

Notably, the addition of 50 mM NaCl decreased the viscosity of high-concentration anti-PD-1 antibody formulations. In contrast, addition of 100 mM NaCl produced equal viscosity reduction, and 140 mM NaCl caused slightly more prominent viscosity reduction effects. As shown in Example 3, Tislelizumab antibody formulated at pH 5.5-6.0 with 50-100 mM NaCl demonstrated best conformation and colloidal stability. Taking into account both viscosity reduction and conformation and colloidal stability, addition of 50-100 mM NaCl in high concentration anti-PD-1 antibody formulations was considered.

TABLE 4
Impact of NaCl and Trehalose on the
Solution Viscosity of High Concentration
anti-PD-1 Antibody Formulations
Containing 20 mM Histidine (pH 6.0)

| | Antibody | Viscosity |
|---|---|---|
| Stabilizer or | Concentration | at 25° C. |
| Viscosity Reducer | (mg/mL) | (cP) |
| None | 160.42 | 16.53 |
| | 173.08 | 29.69 |
| 100 mM trehalose | 143.48 | 13.15 |
| | 164.56 | 23.96 |
| | 180.50 | 31.79 |
| 240 mM trehalose | 151.51 | 16.83 |
| | 163.54 | 21.72 |
| | 178.30 | 36.71 |
| 50 mM NaCl | 155.91 | 13.58 |
| | 182.49 | 26.92 |
| 100 mM NaCl | 151.20 | 12.43 |
| | 187.23 | 31.61 |
| 140 mM NaCl | 158.97 | 14.25 |
| | 178.30 | 25.20 |

The viscosity and osmolality of anti-PD-1 antibody formulations containing 50-100 mM NaCl in presence of trehalose (shown in Table 5) were also studied. The results demonstrated similar viscosity values with 50 mM NaCl and 100 mM trehalose combination having a viscosity of 13.98 cP at 154.70 mg/ml of Tislelizumab. A combination of 70 mM NaCl and 80 mM trehalose had a viscosity of 13.43 cP at 152 mg/ml of Tislelizumab and 100 mM NaCl and 70 mM trehalose had a viscosity of 11.52 cP at 153.64 mg/ml of Tislelizumab.

When the antibody concentration reached about 180 mg/ml, the viscosity values were all about 30 cP. In terms of viscosity, all of the formulations tested demonstrated improved syringe-ability. Specifically, the viscosity generated by high concentration Tislelizumab formulations presented good compatibility with a syringe containing a 23- or 25-gauge needle normally used for subcutaneous administration. In addition, the viscosity of 50 mM NaCl and 100 mM trehalose (33.19 cP at 184.51 mg/ml antibody concentration) combination was higher than 50 mM NaCl alone (26.92 cP at 182.49 mg/mL), indicating that the addition of trehalose of no more than 100 mM was considered based on both viscosity and osmolality considerations.

TABLE 5
The Effect of Combinations of NaCl and Trehalose on the Solution
Viscosity and Osmolality of High Concentration Anti-PD-1 Antibody
Formulations Containing 20 mM Histidine (pH 6.0)

| | Antibody | Viscosity | |
|---|---|---|---|
| Stabilizer and | Concentration | at 25° C. | Osmolality |
| Viscosity Reducer | (mg/mL) | (cP) | (mOsmol/kg) |
| 50 mM NaCl + | 154.70 | 13.98 | 333 |
| 100 mM trehalose | 174.10 | 23.36 | NA |
| | 184.51 | 33.19 | |
| 70 mM NaCl + | 152.00 | 13.43 | 301 |
| 80 mM trehalose | 166.65 | 21.49 | NA |
| | 182.28 | 29.46 | |
| 100 mM NaCl + | 153.64 | 11.52 | 416 |
| 70 mM trehalose | 161.20 | 15.15 | NA |
| | 181.69 | 26.45 | |

### Example 5: Stability of High Concentration PD-1 Antibody Formulations

The stability of high concentration Tislelizumab antibody was evaluated in the various formulations listed in Table 6. All formulations were prepared in 20 mM Histidine-Histidine HCl buffer at pH 6.0. Formulations F15 and F16 were prepared in 100 mM NaCl and 70 mM trehalose combination, while formulation F17 was prepared in 50 mM NaCl and 100 mM trehalose combination.—In these formulations, the concentration of polysorbate 20 ranged from 0 to 0.8 mg/mL (equivalent to 0.08%).

Tislelizumab was buffer exchanged into 20 mM Histidine-Histidine HCl buffer (pH 6.0) by dialysis to generate Tislelizumab stock solutions. Stock solutions of NaCl and trehalose combinations in 20 mM Histidine-Histidine HCl buffer at pH 6.0 were prepared and spiked into the Tislelizumab stock solutions. Subsequently, samples were concentrated to approximately 150 mg/mL with 30 kDa Amicon Ultra™ centrifugal filters. Formulations with varying concentrations of polysorbate 20 (PS20: 0, 0.2 mg/ml and 0.8 mg/ml) were made by addition of a high concentration PS20 stock solution. Each of the formulated solutions was filtered using a 0.22 μm PES syringe filter and filled into 2 mL ready-to-use glass vials, with a 0.5 mL drug product fill volume.

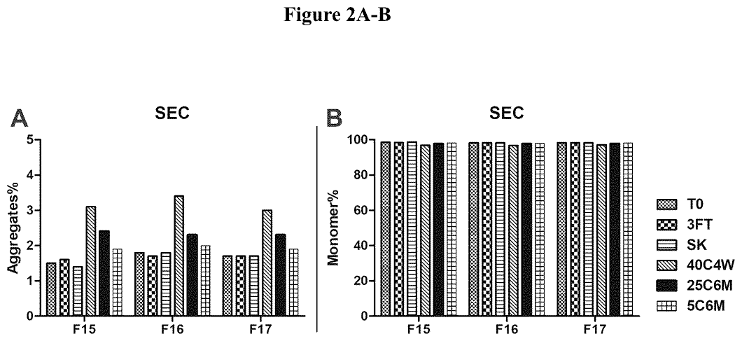

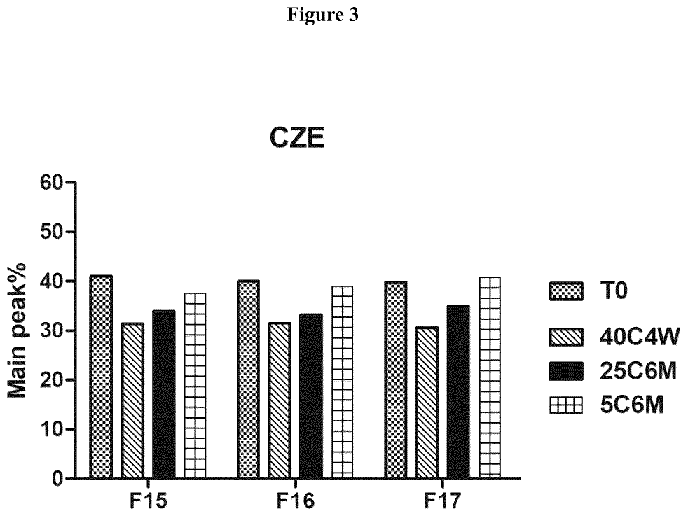

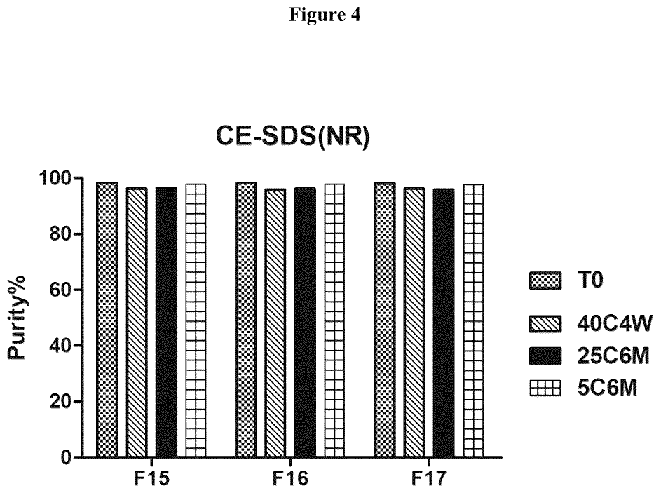

A freeze-thaw study was performed by subjecting the vials to three cycles of freezing at −40° C. and thawing at ambient temperature (noted as “3FT” in graphs). In order to study high temperature stability and agitation stability of formulations, samples were either stored in a 40° C. stability chamber for 4 weeks (noted as “40C4W” in graphs) or were mechanically stressed by agitation for 48-hours (noted as “SK” in graphs). The formulations were evaluated by A350, SEC (purity), CZE (charge profile) and CE-SDS (NR) (purity). As a control, the samples were analyzed at its initial time point, noted as TO in the graphs. These results were provided in Table 8 and FIGS. 2-4.

Turbidity of the drug product was determined by measuring optical density at 350 nm. Samples stored at 40° C. and 25° C. showed a slight increase in turbidity and the A350 of the PS20-free samples were found to be higher than the other samples. Under shaking conditions, a more pronounced increase in A350 was observed in the PS20-free samples, whereas no significant changes were observed in the other samples. There was no measurable change in turbidity upon freeze-thaw stress for any of the formulations.

At 40° C., there was a slight decrease in antibody stability, with a corresponding increase in the amount of aggregates, was observed among formulations F15, F16 and F17. Additionally, there were no significant changes in SEC purity for formulations upon shaking and freeze-thaw stress. The SEC purity of all formulations were within the clinical acceptance criteria of 95.0% for monomer. Samples stored at 40° C. showed a decrease in main peak % and total basic peak % (data not shown), with a corresponding increase in acidic peak % (data not shown). Purity by CE-SDS(NR) indicated that 40° C. storage for 4 weeks resulted only in a slight reduction of monomer. There were no differences among the different formulations at 40° C.

In conclusion, these results demonstrated that high concentration Tislelizumab formulations containing PS20 (e.g., F16 and F17) were stable upon agitation, freeze-thaw and thermal stress, 5° C. and 25° C. storage for 6 months. Subsequently, additional studies were performed to determine the long-term stability and accelerated stability of formulation 18 (F18), which comprised 70 mM NaCl and 80 mM trehalose. Concentrated Tislelizumab drug substance was prepared at about 200 mg/mL in 20 mM Histidine-Histidine HCl buffer, pH 6.0 by concentration and diafiltration. Formulation F18 was prepared by spiking stock solutions of NaCl, trehalose and polysorbate 20 into Tislelizumab drug substance to achieve target compositions listed in Table 6. Each of the formulated solutions was filtered using a 0.22 μm PES syringe filter and filled into 2 mL ready-to-use glass vials, with a 2 mL drug product fill volume. Samples were staged and placed in a 5±3° C. and 25° C. stability chamber with 2 vials for each time point. The planned duration of the study was 24 months at 5±3° C. and 6 months at 25° C.

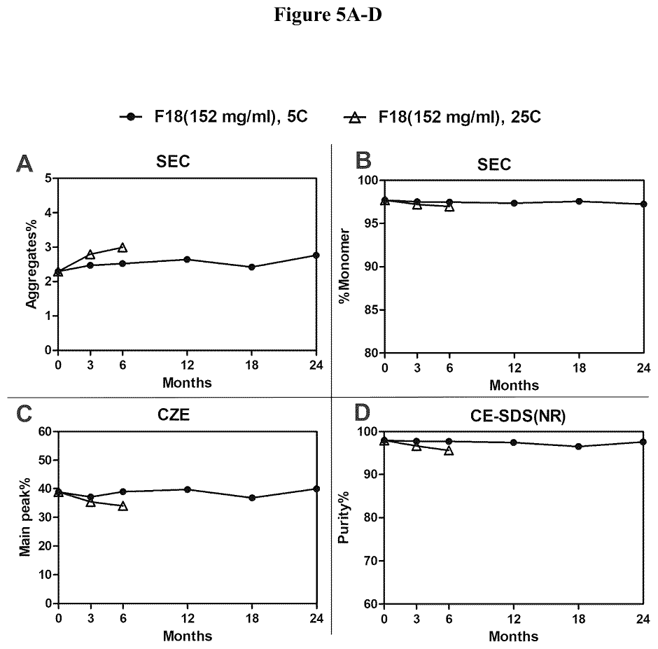

Table 9 summarizes the visible particles and subvisible particles results for up to 24 months for formulation F18 at 152 mg/ml. SEC (purity), CZE (charge profile) and CE-SD S (NR) (purity) results for up to 24 months were presented in FIGS. 5A-D. Formulation F18 at 152 mg/ml was visually inspected and found to be essentially free from visible particles through 24 months at 5±3° C. and 6 months at 25° C. No significant change in subvisible particulates was observed at 5±3° C., and an increase in >10 μm particles at 25° C. was detected. Additionally, no significant change in aggregates, SEC monomer, CZE main peak and CE-SDS(NR) purity at 5±3° C. was detected. Storage for 6 months at 25° C. resulted in a slight increase in aggregates and gave rise to decrease in sample main peak of CZE and CE-SDS(NR) purity. However, the increase in subvisible particles and aggregates and the decrease in main peak of CZE and CE-SDS(NR) purity at 25° C. are acceptable for liquid formulations with intended storage conditions of 5±3° C.

Therefore, in taking all of these results together, formulations F16, F17 and F18 are appropriate subcutaneous anti-PD1 antibody formulations suitable for clinical use. Especially, based on months of stability data, formulation F18 at 152 mg/ml is stable at the recommended storage condition of F1C for up to 24 months with no measurable changes in product quality attributes.

TABLE 6
Formulation Compositions Used in Stability Studies

| | Antibody | |
|---|---|---|
| | Concentration | |
| No. | (mg/mL) | Formulation Composition |
| F15 | 157.97 | 20 mM Histidine-Histidine HCl, 100 mM NaCl + |
| | | 70 mM trehalose, pH6.0 |
| F16 | 157.97 | 20 mM Histidine-Histidine HCl, 100 mM NaCl + |
| | | 70 mM trehalose, 0.08% polysorbate 20, pH6.0 |
| F17 | 151.02 | 20 mM Histidine-Histidine HCl, 50 mM NaCl + |
| | | 100 mM trehalose, 0.02% polysorbate 20, pH6.0 |
| F18 | 152 | 20 mM Histidine-Histidine HCl, 70 mM NaCl + |
| | | 80 mM trehalose, 0.08% polysorbate 20, pH6.0 |

TABLE 7
Formulation Stability Studies Scheme

| | Study Conditions | Test Items |
|---|---|---|
| High temperature | Samples were placed in a 40° C. | Turbidity (OD₃₅₀ₙₘ) |
| (40° C.) study | stability chamber for 4 weeks | Purity (SEC and |
| Agitation study | Agitation by shaking, 800 rpm, 48 hours | CE-SDS(NR)) |
| Freeze-thaw | Three cycles of freezing at −40° C. | Charge variants (CZE) |
| study | and thawing at ambient temperature. | |
| Accelerated | Samples were placed in a 25° C. | Visible particles |
| stability study | stability chamber for 6 months | Subvisible particles (MFI) |
| (25° C.) | | Purity (SEC and |
| Long-term | Samples were placed in a 5 ± 3° C. | CE-SDS(NR)) |
| stability study | stability chamber for 24 months | Charge variants (CZE) |
| (5 ± 3° C.) | | |

TABLE 8
Turbidity results for Formulations F15, F16 and F17

| | | Ini- | | | | | |
|---|---|---|---|---|---|---|---|
| | | tial | 40C4W | Shaking | 3FT | 25C6M | 5C6M |
| Turbidity | F15 | 0.106 | 0.141 | 0.485 | 0.107 | 0.135 | 0.104 |
| (OD_{350 nm}) | F16 | 0.104 | 0.130 | 0.104 | 0.103 | 0.126 | 0.118 |
| | F17 | 0.093 | 0.125 | 0.104 | 0.104 | 0.116 | 0.105 |

Notes:
40C4W refers to the samples stored at 40° C. for 4 weeks. 3FT refers to the samples subjected to 3 freeze/thaw cycles. 25C6M refers to the samples stored at 25° C. for 6 months. 5C6M refers to the samples stored at 5° C. for 6 months.

TABLE 9
Stability data for Formulation F18 at 5° C. and 25° C.

| | | | | 3 | 6 | 12 | 18 | 24 |
|---|---|---|---|---|---|---|---|---|
| | Time Interval | | Initial | months | months | months | months | months |
| 152 | Visible particles | | VPF | VPF | VPF | VPF | VPF | VPF |
| mg/ml, | Subvisible | ≥10 μm | 35 | 89 | 85 | 15 | 40 | 49 |
| 5 C. | particles | ≥25 μm | 7 | 20 | 6 | 2 | 2 | 1 |
| | (particle/ml) | | | | | | | |
| 152 | Visible particles | | VPF | VPF | VPF | NA | NA | NA |
| mg/ml, | Subvisible | ≥10 μm | 35 | 564 | 242 | NA | NA | NA |
| 25 C. | particles | ≥25 μm | 7 | 31 | 25 | NA | NA | NA |
| | (particle/ml) | | | | | | | |

Notes:
VPF refers to visible particles free. 5 C. refers to the samples stored at 5 ± 3° C. 25 C. refers to the samples stored at 25° C.

### Example 6: Bioavailability of Subcutaneous Injection

In order to test the bioavailability of the subcutaneous formulation, formula 18 (F718, Table 6) was used. Tislelizumab was administered intravenously (i.v.) to mice and compared with 10 mpk (mg per kg) F18 subcutaneous administration into the back of the animal, 10 mpk subcutaneous administration into the abdomen of the animal or 20 mpk subcutaneous administration to the abdomen of the animal. For this study C57 mice with a knock in of human PD1 were used. The bioavailability of subcutaneous injection overall was 75.800. This data is shown in Table 10 below.

TABLE 10
Subcutaneous administration of Tislelizumab into C57-hPD1 mice

| | i.v | s.c-B | s.c-A | s.c-A | i.v | s.c-B | s.c-A | s.c-A |
|---|---|---|---|---|---|---|---|---|
| Dose | 10 | 10 | 10 | 20 | 10 | 10 | 10 | 20 |
| (mg/kg) | | | | | | | | |
| Conc. | 2 | 1 | 1 | 2 | 2 | 1 | 1 | 2 |
| (mg/mL) | | | | | | | | |
| | AUC₀₋ₗₐₛₜ (hr*μg/mL) | | | | Bioavailability (%) | | | |
| #1 | 10819 | 8691 | 5886 | 12666 | / | 91.2 | 61.8 | 66.4 |
| #2 | 7764 | 6909 | 9026 | 15054 | / | 72.5 | 94.7 | 79.0 |
| #3 | 10009 | 7231 | 7494 | 14426 | / | 75.9 | 78.6 | 75.7 |
| #4 | | 6800 | 7840 | 12451 | / | 71.4 | 82.3 | 65.3 |
| Mean | 9531 | 7408 | 7561 | 13649 | / | 78 | 79 | 72 |
| Median, | | | | | / | 75.8% [95% CI: 69.9-82.5%] | | |
| 95% CI | | | | | | | | |

Note:
s.c-B, subcutaneous injection on the Back;
s.c-A, subcutaneous injection at Abdomen

In a different study, NOD-SCTD mice were used. Tislelizumab was administered intravenously (i.v.) to mice and compared with 10 mpk (mg per kg) F18 subcutaneous administration into the back of the animal, 20 mpk subcutaneous administration to the back of the animal, 10 mpk subcutaneous administration into the abdomen of the animal or 20 mpk subcutaneous administration to the abdomen of the animal. The bioavailability of subcutaneous injection overall was 54.8%. This data is shown in Table 11 below.

TABLE 11
Subcutaneous administration of Tislelizumab into NOD-SCID mice

| | i.v | s.c-B | s.c-B | s.c-A | s.c-A | i.v | s.c-B | s.c-B | s.c-A | s.c-A |
|---|---|---|---|---|---|---|---|---|---|---|
| Dose | 10 | 10 | 20 | 10 | 20 | 10 | 10 | 20 | 10 | 20 |
| (mg/kg) | | | | | | | | | | |
| Conc. | 2 | 1 | 2 | 1 | 2 | 2 | 1 | 2 | 1 | 2 |
| (mg/ml) | | | | | | | | | | |
| | AUC₀₋ₗₐₛₜ (hr*μg/mL) | | | | | Bioavailability (%) | | | | |
| #1 | 12187 | 6662 | 13593 | 8798 | 10385 | / | 57.5 | 58.7 | 76.0 | 44.9 |
| #2 | 11105 | 8117 | 12390 | 6354 | 11318 | / | 70.1 | 53.5 | 54.9 | 48.9 |
| #3 | 11499 | 8820 | 12640 | 8605 | 11257 | / | 76.2 | 54.6 | 74.3 | 48.6 |
| #4 | 11515 | 7085 | 12321 | 6329 | 12127 | / | 61.2 | 53.2 | 54.7 | 52.4 |
| Mean | 11577 | 7671 | 12736 | 7521 | 11272 | / | 66 | 55 | 65 | 49 |
| Median, | | | | | | / | 54.8% [95% CI: 53.4-64.1%] | | | |
| 95% CI | | | | | | | | | | |

Note:
s.c-B, subcutaneous injection on the Back;
s.c-A, subcutaneous injection at Abdomen

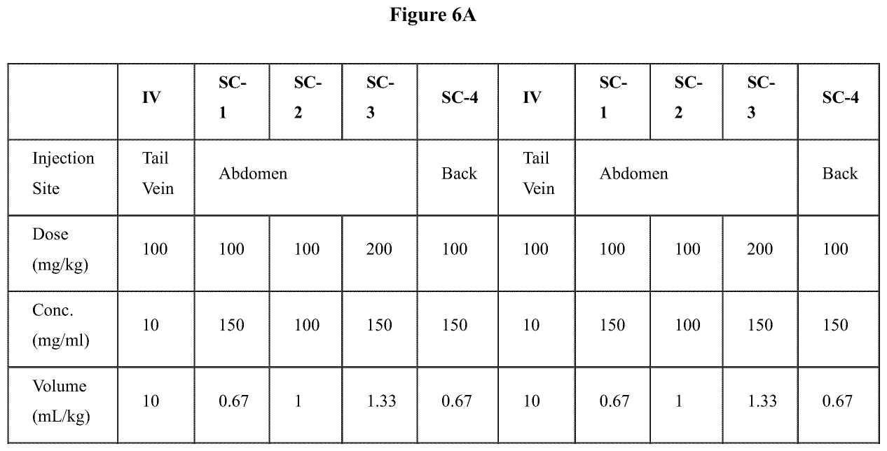

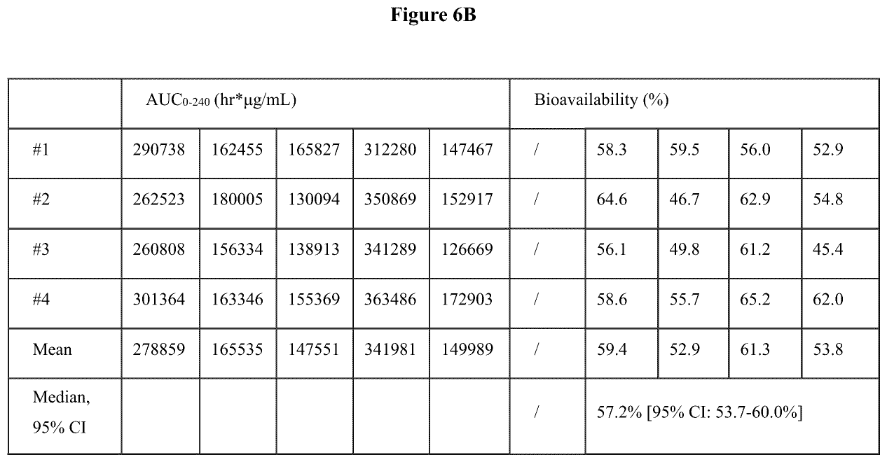

The F18 formulation was also tested in a larger animal, that of Sprague Dawley rats. Tislelizumab was administered intravenously at 100 mpk (10 mg/ml), subcutaneously into the abdomen of the rat at 100 mpk (150 mg/mi), subcutaneously into the abdomen at 100 mpk (100 mg/ml), subcutaneously into the abdomen at 200 mpk (150 mg/ml), or subcutaneously into the back at 100 mpk (150 mg/ml). The overall bioavailability was 57.2%, and this data is shown in FIG. 6A-B.

The F18 Tislelizumab formulation was also tested in a minipig model. For this study, 6 mpk was administered i.v. to the minipig, while 6 mpk was injected subcutaneously into the leg or 6 mpk was injected subcutaneously behind the ear of the animal. Overall bioavailability of Tislelizumab by subcutaneous injection was 79.8%. This data is shown in Table 12.

TABLE 12
Subcutaneous administration of Tislelizumab into minipigs

| | | | SC- | | | SC- |
|---|---|---|---|---|---|---|
| | | SC- | Behind | | SC- | Behind |
| | IV | Leg | Ear | IV | Leg | Ear |
| Injection | Intravenous | Leg | Behind | Intravenous | Leg | Behind |
| Site | bolus | | ear | bolus | | ear |
| Dose | 6 | 6 | 6 | 6 | 6 | 6 |
| (mg/kg) | | | | | | |
| Conc. | 10.3 | 147.3 | 147.3 | 10.3 | 147.3 | 147.3 |
| (mg/ml) | | | | | | |
| Volume | 0.583 | 0.041 | 0.041 | 0.583 | 0.041 | 0.041 |
| (mL/kg) | | | | | | |
| | AUC₀₋₃₃₆ (hr*μg/mL) | | | Bioavailability (%) | | |
| #1 | 15220.45 | 7613.11 | 10915.52 | / | 58.03 | 83.20 |
| #2 | 7584.392 | 10022.78 | 5544.97 | / | 76.40 | 42.27 |
| #3 | 16552.22 | 13093.80 | 7899.81 | / | 99.81 | 60.22 |
| #4 | / | 14910.93 | 8426.25 | / | 113.66 | 64.23 |
| #5 | / | 14466.60 | 14982.78 | / | 110.27 | 114.21 |
| Mean | 13119.02 | 12021.44 | 9553.87 | / | 91.63 | 72.83 |

| | | |
|---|---|---|
| Median, | / | 79.8% [95% CI: |
| 95% CI | | 63.6-100.9%] |

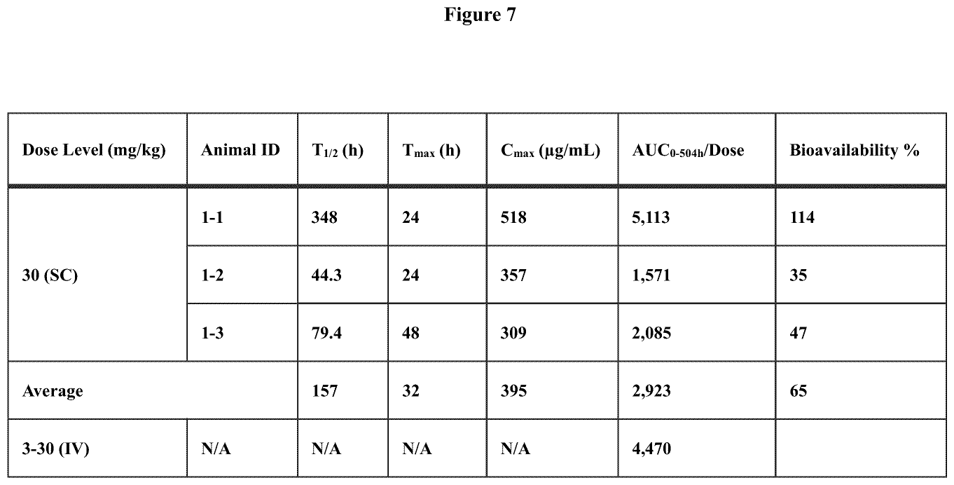

Finally, the Tislelizumab subcutaneous formulations were tested in a monkey PK study. Three monkeys were dosed with 30 mg/kg of the F18 Tislelizumab formulation, with blood being collected at timepoints 0, 4 hours, 8 hours, 24 hours, 48 hours, 4 days, 7 days, 14 days, and 21 days post dose. The results are shown in FIG. 7, with the subcutaneous result compared to an AUC of a previously conducted monkey study using the IV formulation and administration. There were no apparent changes in body weight and no clinical pathology was noted. No histopathology changes were noted at the injection site.

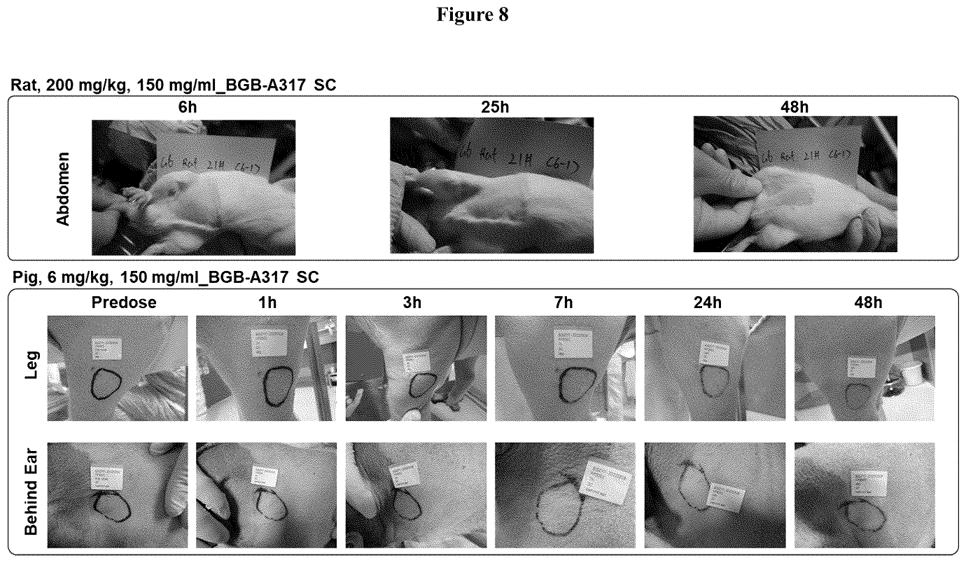

The Tislelizumab subcutaneous formulation was well tolerated, with no injection site reaction either in the rat or minipig models regardless of injection site or concentration used as is shown in FIG. 8.

1. A low viscosity pharmaceutical formulation comprising:
about 10 mg/mL to about 200 mg/mL of an anti programmed death receptor 1 (PD-1) antibody, or antigen binding fragment thereof;
a formulation buffer providing a pH of about 5.0 to about 7.0;
a sugar polyol;
a viscosity reducer; and
a non-ionic surfactant,
wherein the pharmaceutical formulation has a viscosity of no more than 30 cP, and an osmolarity from about 200 mOsmol/kg to about 400 mOsmol/kg.

2. The formulation of claim 1, wherein the PD-1 antibody or antigen binding fragment thereof, comprises a heavy chain variable region (a) a HCDR1 (Heavy Chain Complementarity Determining Region 1) of SEQ ID NO: 1, (b) a HCDR2 of SEQ ID NO:2, (c) a HCDR3 of SEQ ID NO:3 and a light chain variable region that comprises: (d) a LCDR1 (Light Chain Complementarity Determining Region 1) of SEQ ID NO:4, (e) a LCDR2 of SEQ ID NO:5, and (f) a LCDR3 of SEQ ID NO:6.

3. The formulation of claim 2, wherein the formulation buffer is:
(a) selected from the group consisting of histidine, acetate, citrate, succinate, phosphate, mixture of histidine and acetic acid, or mixture of histidine and citric acid; or
(b) histidine.

4. (canceled)

5. The formulation of claim 3, wherein the concentration of buffer is 15 mM to 25 mM.

6. The formulation of claim 5, wherein;
(a) the formulation comprises 20 mM histidine buffer;
(b) the pH is 5.5-6.0; or
(c) a combination thereof.

7. (canceled)

8. The formulation of claim 1, wherein the sugar polyol is:
(a) selected from the group consisting of trehalose, sucrose, sorbitol, mannitol, maltose, dextran, or (2-hydroxypropyl)-β-cyclodextrin, or
(b) trehalose.

9. (canceled)

10. The formulation of claim 8, wherein the trehalose concentration is:
(a) from 70 mM to 240 mM;
(b) from 80 mM to 160 mM;
(c) from 70 mM to 100 mM; or
(d) 80 mM.

11. (canceled)

12. (canceled)

13. (canceled)

14. The formulation of claim 1, wherein the viscosity reducer is an inorganic salt selected from the group consisting of sodium chloride, magnesium chloride, calcium chloride, sodium acetate, sodium sulfate, ammonium chloride or ammonium sulfate.

15. The formulation of claim 14, wherein the inorganic salt is sodium chloride at a concentration of 50 mM to 150 mM,

16. The formulation of claim 15, wherein the sodium chloride is at a concentration of:
(a) 50 mM to 100 mM, or
(b) 70 mM.

17. (canceled)

18. The formulation of claim 1, wherein the non-ionic surfactant is selected from the group consisting of polysorbate 20, polysorbate 80 or poloxamer 188.

19. The formulation of claim 18, wherein the concentration of polysorbate 20 is:
(a) from 0.02% to 0.08%, or
(b) 0.08%.

20. (canceled)

21. The formulation of claim 1, wherein the formulation comprises:
(a) 20 mM Histidine-Histidine HCl, 100 mM NaCl, 70 mM trehalose and 0.08% polysorbate 20, with a pH of pH6.0;
(b) 20 mM Histidine-Histidine HCl, 50 mM NaCl, 100 mM trehalose and 0.02% polysorbate 20, with a pH of pH6.0; or
(c) 20 mM Histidine-Histidine HCl, 70 mM NaCl, 80 mM trehalose and 0.08% polysorbate 20 with a pH of pH6.0.

22. (canceled)

23. (canceled)

24. The formulation of claim 1, wherein the concentration of the anti-human PD-1 antibody, or antigen binding fragment thereof is from about 100 mg/mL to 200 mg/mL.

25. A method of making an antibody formulation, the method comprising:
(a) adding trehalose and sodium chloride to the antibody to achieve an antibody formulation having a concentration of trehalose no less than 50 mM and a concentration of sodium chloride no less than 25 mM, wherein the antibody comprises a heavy chain variable region (a) a HCDR1 (Heavy Chain Complementarity Determining Region 1) of SEQ ID NO: 1,
(b) a HCDR2 of SEQ ID NO:2, (c) a HCDR3 of SEQ ID NO:3 and a light chain variable region that comprises: (d) a LCDR1 (Light Chain Complementarity Determining Region 1) of SEQ ID NO:4, (e) a LCDR2 of SEQ ID NO:5, and (f) a LCDR3 of SEQ ID NO:6;
(b) concentrating the antibody formulation of (a) from 150-200 mg/mL; and
(c) adding polysorbate 20 to the antibody formulation of (b) to obtain an antibody formulation having a concentration of polysorbate 20 of no less than 0.01 mg/mL,
wherein the antibody formulation of (c) is in an aqueous solution and has a viscosity of no more than 30 cP at 25° C., and wherein the antibody formulation of (c) is stable upon agitation, freeze-thaw and thermal stress.

26. A method for treating cancer in a human patient in need thereof comprising subcutaneous administration of an effective amount of an anti-human PD-1 antibody formulation of claim 1.

27. The method of claim 26, wherein the anti-human PD-1 antibody formulation is subcutaneously administered at a dose of about 100 mg to about 1000 mg.

28. The method of claim 27, wherein the anti-human PD-1 antibody formulation is subcutaneously administered:
(a) at a dose of 200 mg;
(b) at a dose of 300 mg;
(c) at a dose of 400 mg;
(d) at a dose of 500 mg;
(e) once a week;
(f) once every 2 weeks;
(g) once every 3 weeks; or
(h) any combination thereof.

29. (canceled)

30. (canceled)

31. (canceled)

32. (canceled)

33. (canceled)

34. (canceled)

35. The method of claim 26, wherein the cancer is lung cancer, small-cell lung cancer, non-small cell lung cancer, adrenal cancer, liver cancer, stomach cancer, cervical cancer, melanoma, renal cancer, breast cancer, colorectal cancer, leukemia, bladder cancer, bone cancer, brain cancer, an endometrial cancer, head and neck cancer, lymphoma, ovarian cancer, skin cancer, thyroid tumor, or esophageal cancer.

36. The method of claim 26, wherein the human patient is administered at least one other therapeutic agent.

37. The method of claim 36, wherein the at least one other therapeutic agent is zanubrutinib, pamiparib, an anti-CTLA4 antibody, an anti-4-1BB antibody, an anti-OX40 antibody, an anti-TIGIT antibody, an anti-TIM-3 antibody, a second PD-1 antibody, a CD40 agonist, a TLR agonist, a CAR-T cell, or a chemotherapeutic agent.
